(19) United States
(12) Patent Application Publication
(10) Pub. No.: US 2025/0316329 A1
(43) Pub. Date: Oct. 9, 2025

(54) MULTICAPITATE TRANSFORMERS FOR AI-BASED PROTEIN AND DRUG DESIGN

(71) Applicant: Stephen Gbejule Odaibo, Sugar Land, TX (US)

(72) Inventor: Stephen Gbejule Odaibo, Sugar Land, TX (US)

(73) Assignee: Deep EigenMatics LLC, Sugar Land, TX (US)

(21) Appl. No.: 19/093,021

(22) Filed: Mar. 27, 2025

(51) Int. Cl. G16B 15/30; G16B 30/20; G16B 40/20

(52) U.S. Cl. CPC G16B 15/30; G16B 30/20; G16B 40/20

(57) ABSTRACT

Methods and apparatus for determining protein and ligand sequence, structure, and docking site given a target protein sequence and structure are presented. A multicapitate transformer architecture with a number of heads including a sequence head and a structure head is introduced, wherein given a target protein sequence and structure, a candidate ligand is generated, wherein the transformer's sequence head yields the ligand sequence and the structure head yields the ligand structure and docking site. Non-capitate weights are shared between the output heads. In one embodiment, a discriminative feature localization method is used to optimize the target protein's input structure representation towards the desired ligand effect class. The methods and apparatus presented enable design and synthesis of both peptide ligands and small molecule drugs each with specified ligand effect categories.

### FIELD OF THE INVENTION

The present invention relates generally to Artificial Intelligence (AI) and Machine Learning (ML) methods for protein and drug design, and specifically to the use of transformer architectures for protein and drug design.

### BACKGROUND OF THE INVENTION

Novel and effective drugs are difficult and expensive to develop. As a result, there are a plethora of diseases for which available therapies are either non-existent or grossly ineffective. The exorbitant cost and duration of new drug discovery and development is part of the problem, and it often costs over $2 billion and more than 10 years to get a single candidate drug through clinical testing phases, only for a high percentage of candidate drug to fail in the clinical testing phases, despite the tremendous time and resource investment. One critical reason for the high failure rate is a general lack of adequate specificity in the design and development of the drugs. In particular, cell signaling depends exquisitely on ligand sequence, structure, and docking site on target proteins. Furthermore, these three defining features of ligand function—sequence, structure, and docking site—are inextricably linked. Therefore the problem of designing a ligand for a given target protein must utilize methods that integrate the determination of these intrinsically linked features. Existing methods however fall short in this regard, and this has created a significant unmet need for more powerful and integrated methods.

In recent years, however, deep learning has introduced a wide array of techniques into the field of protein design and structure determination; and these methods have significantly advanced the field. Nonetheless, the aforementioned unmet need has not yet been addressed by existing deep learning methods.

The standard transformer architecture was introduced in 2017 (See Vaswani, Ashish, et al. “Attention is all you need.” Advances in Neural Information Processing Systems 30 (2017)), and has essentially revolutionized the field of natural language processing. In particular, the transformer architecture template serves as the backbone of essentially all large language models to date.

Since the transformer's introduction in 2017, there have been several instances in which it has been used to address the protein folding problem and related problems. The standard transformer architecture has only one final output head. It is unicapitate. Here in the Specifications as well as in the Claims, by “final output head” or “transformer head,” we mean the aspect of the architecture where the loss function value is computed during training and where the inference value(s) are determined during inference. Of note, this is generally distinct from the head of an attention block or module as used in the term “multi-headed attention,” which is simply a parallelization implementation module of the attention mechanism.

In the invention disclosed herein, we introduce a multicapitate (“multiple headed”) transformer for ligand design, i.e. for the purpose of determining the sequence, structure, and docking site of a ligand given the sequence and structure of a target protein. In this invention, the multicapitate transformer includes two final output heads: a sequence head and a structure head. The sequence head yields the ligand sequence while the structure head yields the ligand structure. Furthermore, the non-capitate weights of the architecture are shared in the sense that backpropagation of errors from any one of the heads updates all weights downstream from the head terminal that contributed the loss computed at that particular head terminal.

This multicapitate architecture enables for fully integrated joint learning of sequence, structure, and docking site of a ligand given a target protein sequence and structure; and therefore increases the likelihood of yielding novel drug ligands that can effectively treat diseases.

Prior to the invention disclosed herein, there were no multicapitate transformer architectures for protein and drug design, wherein a structure head and a sequence head were operative such that the non-capitate weights were shared to enable sequence, structure, and docking site to be jointly learned at once. More so, prior to this disclosure, there were no such multicapitate transformers for protein and drug design equipped with a discriminative feature localization for refinement of target protein structure input towards desired ligand class optimization.

Generally, both target proteins and their associated ligands can separately assume a number of structural conformations. Their respective conformation when in a bound state complex restricts the range of the conformational states. Nonetheless they do not assume a unique or static conformation even when in a complex bound state. It is therefore essential to determine the structure of the ligand using a method highly cognizant of its associated target protein's sequence and structure. In particular, ligand sequence and structure should ideally be determined simultaneously, integrally, and in a manner directly conditioned on the target protein's conformational structure when in a bound state complex with the ligand.

The multicapitate transformer introduced in this invention disclosure accomplishes this objective.

Conversely, the target protein can also assume a range of structural conformations in general. It is therefore essential to use a conformational structure representation of the target protein optimized towards the desired ligand effect class. For instance, if one seeks to determine an agonist of a given target receptor, an agonist bound conformation of that receptor is more rational than an antagonist bound or an unbound conformation. As such, the target protein structure representation must also be optimized towards a conformation consistent with the desired ligand effect class.

A localized input structure refinement method guided by discriminative feature localization mapping accomplishes this objective. Together, the combination of a multicapitate transformer, as introduced in this invention, and a discriminative feature localization-guided structure refinement is a natural one.

This invention thereby increasing the likelihood of yielding novel and effective drugs and therapies for diseases.

### OBJECTS OF THE INVENTION

It is an object of this invention to provide a system, method, and apparatus for peptide ligand sequence, structure, and docking site determination using a multicapitate transformer architecture with a separate sequence head and a separate structure head, wherein the non-capitate weights are shared.

Another object of this invention is to provide a system, method, and apparatus for small molecule drug identity and docking site determination using a multicapitate transformer architecture with a separate sequence head and a separate structure head, wherein the non-capitate weights are shared.

Yet another object of the invention is providing the aforementioned multicapitate transformers wherein they are each equipped with a discriminative feature localization mechanism used to optimize the target protein input structure representation towards a conformation consistent with the specified ligand effect category.

Yet other objects, advantages, and applications of the invention will be apparent from the specifications and drawings included herein.

### SUMMARY OF THE INVENTION

The invention disclosed herein includes a method to use a multicapitate (“multiple headed”) transformer architecture to obtain the sequence, structure, and docking site of a candidate ligand, given only a target protein sequence and structure and a desired ligand effect class. The candidate ligand may be a peptide ligand or a small molecule drug (SMD). In the case of an SMD, the sequence length is taken to be 1.

The multicapitate transformer includes two heads, a sequence head which yields the ligand's sequence, and a structure head which yields the ligand's structure as well as its docking site on the given target protein.

As used here and in the claims, the term “transformer head” or “final output head” means the aspect of the transformer architecture where the loss function value is computed during training and where the inference value(s) are determined during inference. In particular, as used here and in the claims, the term transformer head or “final output head,” do not refer to the “attention heads” of multi-headed attention, which is a parallelization implementation of the attention mechanism. The number of layers in any given head of a transformer is an architectural design hyperparameter.

As used here and in the claims, the term “transformer” means a neural network that includes an attention mechanism. The precise implementation and applications of transformer architectures are myriad. By way of example and not limitations, transformers may include (or not include) skip connections, feed forward layers, linear layers, position encodings, multi-headed implementations of attention mechanism (“multi-headed attention”), or cross-attention layers. Furthermore, in general, the architecture category of a transformer may be one of encoder-decoder, encoder-only, or decoder-only.

The term “attention mechanism” refers to a learnable means of determining how much (or how little) influence or attention to differentially allocate to individual tokens in a context array when transforming a given token. In one embodiment, the allocation weights are values of a probability distribution over the tokens in the context array. An embodiment of an attention mechanism, scaled dot product attention, is defined below in the detailed descriptions of preferred embodiments.

The invention disclosed herein further includes a method comprising preparing or accessing a database of target protein-ligand complexes (or target proteins and corresponding ligands in bound state conformations), wherein the database is segmented or indexed in a signaling pathway-specific manner. By database we mean a diverse plurality of target protein-ligand complexes or a diverse plurality of target proteins and corresponding ligands in complex state conformations.

For instance, by way of example and not limitation, such a database may include the G-protein Coupled Receptor (GPCR), Angiotensing II Type 1 receptor (AT1R) in complex with the peptide ligand, angiotensin II. The corresponding effect label (or index) of the complex would be ‘agonist,’ and the associated signaling pathway based on which angiotensin is an agonist must be specified. In this case, it is a G_{q/11} and Gio mediated pathway.

The signaling pathway specific database of target protein-ligand complexes is used to train a Signaling Pathway Specific Discriminative Classifier (SPS-DC) neural network. The SPS-DC neural network is configured to accept target protein sequence and structure as input, and as output it yields a classification into a ligand effect category. By way of example but not limitation, the ligand effect category could be agonist-bound conformation, unbound conformation, or antagonist-bound conformation.

Furthermore, the SPS-DC neural network is equipped with a discriminative feature localization mechanism whereby it outputs a feature map specifying the discriminative features of the target protein. For example, if a given target protein is classified as agonist-bound at SPS-DC inference time, the discriminative feature localization mechanism will indicate which features of the target protein caused the SPS-DC neural network to classify it as agonist-bound.

The discriminative feature localization mechanism can be any method that enables localization of the particular features in the target protein sequence and structure representation that decided the class. By way of example and not limitation, discriminative feature localization methods include Class Activation Mapping (CAM) and CAM-variants. As used in this description, the term CAM-variant means any method that uses a decomposition of the neural network's feature extraction, weighted scalings, and activations to determine the discriminative feature map. Examples of CAM-variants include but are by no means limited to Gradient-weighted Class Activation Mapping (Grad-CAM), Guided Grad-CAM, Guided Backpropagation, Integrated Gradients, Eigen-CAM, Self-Matching CAM, Grad-CAM++, Smooth Grad-CAM++, Score CAM, Ablation-CAM, Layer-wise Relevance Propagation (LRP), and Shap-CAM.

Another type of method of discriminative feature localization is occlusion sensitivity analysis.

In one embodiment of the invention, the discriminative feature localization method is a Class Activation Map (CAM). These may use a Global Average Pooling (GAP) step following a series of feature extraction steps. In particular, given a target protein representation as input, the SPS-DC layers serve as feature extractors yielding a set of feature maps. Each feature map can be condensed into a single scalar via a global average pooling operation, for instance. Together, the set of feature maps therefore becomes a feature vector after the GAP operation. The feature vector may be connected via a densely connected (“Fc7”) layer to an output node activated by a Rectified Linear Unit (ReLU) or similar activation function. This output in turn can be passed into a softmax activation so as to generate a probability distribution as the final output. Since the ReLU family of activations are monotonically increasing over positive input domain and zero otherwise, it follows that classification into a given class occurs when scaled inputs from the feature vector are positive. This in turn occurs when the scaled feature maps are positive and higher than those of the non-selected class. The scaled feature maps can be upsampled and overlaid on the input target protein structure representation to identify the aspects of the structural parameters and sequence that determined the classification.

Upon identifying the discriminative features, the next step is to pass the target protein's structure representation and associated discriminative feature map as input into a Localized Structure Update Engine. This yields an updated protein structure. In one embodiment, only the discriminative feature maps are changed from the input structure. Furthermore, at convergence, the updated structure is optimized towards the desired ligand effect class.

The Localized Structure Update Engine consists of the SPS-DC neural network as well as a localized structure update method. The localized structure update method could be any number of methods including but not limited to stochastic gradient descent (SGD) and variants, genetic algorithms and variants, particle swarm optimization methods and variants, and simulated annealing and variants. As noted, in some embodiments it could be a genetic algorithm whereby the SPS-DC evaluates and checkpoints the ligand effect classification following a certain number of iterations. A similar checkpointing forward-facing approach can be applied to particle swarms with the trained SPS-DC as value function.

The signaling pathway-specific database of target protein-ligand complexes is further segmented by ligand effect class. For instance, one segment could contain only receptor-agonist complexes, another segment could contain only receptor-antagonist complexes, and so on.

The segmented database is then used to train an expert transformer of multicapitate architecture. It is expert in the sense that each such multicapitate transformer is specialized in the ligand effect segment category of its training dataset. For example, one expert transformer's expertise would be in the design of agonist peptide ligands, another expert transformer's expertise would be in the design of antagonist peptide ligands, and so on; wherein the respective training datasets are of receptor-agonist complexes, receptor-antagonist complexes, and so on.

In one embodiment of the invention, at inference time each expert transformer neural network is equipped with a CAM-guided structure refinement engine, each of which contains a trained SPS-DC neural network as a main component.

At inference time, the structure input (a vector of structure parameters) is first refined by the CAM-guided structure refinement engine according to the requisite ligand effect classification. The refined structure input is then passed into a structure embedding to yield a structure embedding vector. The weights of the structure embedding are learnable parameters of the multicapitate transformer neural network.

The structure input is a vector of structure parameters. In one embodiment, the structure input is of fixed length, whereby for target proteins whose sequence length are below the structure input length, the unfilled entries are padded with zeros. The structure input fixed length is a hyperparameter of the system. Factors that may determine the choice include the distribution of sequence lengths of proteins in the human body or of known industrial enzymes. The largest known cell surface receptor in the human body for instance is Very Large G-protein Coupled Receptor 1b (VLGP CR 1b) with 6307 amino acids. For example, in some embodiments specifically for designing ligands for human cell surface receptor target proteins, one may set a structure input fixed length upper bound of around n*6307, where n is the number of structure parameters per residue in the chosen structure representation.

In the invention disclosed herein, the transformer architecture residue embeddings are a separately trained neural network. The trained residue embedding is then plugged into the transformer architecture both during training and inference of the transformer. The residue embedding weights, however, are not learnable parameters of the transformer.

In one embodiment of the invention, the residue embedding is trained using a loss function that enforces the following: inner products of embeddings of amino acid residues that are generally further apart—in protein sequences—should be closer to zero, while inner products of embeddings of amino acid residues that are generally closer to each other—in protein sequences—should be closer to one. The general proximity of amino acid residues to each other is inferred from the plurality of protein representations in a residue embedding training database.

In the invention disclosed herein, there is a difference in the handling of ligand peptide design and the handling of small molecule drug ligand design. For the task of peptide ligand design as outlined in this disclosure, i.e. the task of for a given target protein, obtaining a peptide ligand sequence, structure, and docking site, an autoregressive procedure is used during training and inference. However, for the task of small molecule drug (SMD) ligand design as outlined in this disclosure, i.e. the task of for a given target protein, obtaining an SMD identity and docking site, the output is taken as being of sequence length one, hence autoregression is not used.

A number of embodiments arise based on autoregression implementation in the case of peptide ligand design, as outlined in this disclosure. This invention introduces a multicapitate transformer architecture, wherein a sequence head yields the sequence and a structure head yields the structure, each returned residue-wise in an autoregressive manner. As the autoregression procedure progresses, the self-attention context array of tokens is updated in each iteration of the autoregression by adjoining the array with some output of the previous iteration. In one embodiment, only the sequence output (i.e. the residue embedding) is adjoined to the context array. In another embodiment, both the sequence output and the structure output (i.e. structure embedding) are adjoined to the self-attention context array.

There are a number of approaches via which the emerging ligand's structure can be attended to during the training process. In one such embodiment, the thus-far-determined ligand (i.e. the emerging peptide ligand) is itself a peptide, and can therefore be acted on by a structure embedding procedure to yield its corresponding structure embedding vector. This structure embedding vector can then simply be updated as the emerging peptide ligand grows. The emerging peptide ligand's structure embedding weights are learnable parameters of the transformer and in one embodiment are a separate set of weights from the target protein's structure embedding weights.

In summary, the invention disclosed herein consists of systems, methods, and apparatus using multicapitate transformers equipped with discriminative feature localization mechanisms to obtain and synthesize effective peptide ligands or small molecule drug ligands for given target proteins, wherein the target protein's sequence and structure representation is given, and wherein the desired effect category of the peptide ligand or small molecule drug is specified, and wherein the high structural specificity of target protein-driven cellular signaling is properly accounted for as is the inextricable linkage of sequence and structure.

The invention consists of several outlined processes below, and their relation to each other, as well as all modifications which leave the spirit of the invention invariant. The scope of the invention is outlined in the claims section.

### BRIEF DESCRIPTION OF THE DRAWINGS

In the following detailed description of the invention, we reference the herein listed drawings and their associated descriptions, in which:

FIG. 1 is an illustrative example of protein structure with voxel-wise amino acid residue probability representation.

FIG. 2 is an illustrative example of associated probability distributions of voxels in voxel-wise amino acid residue probability representation of protein structure.

FIG. 3 is an illustrative example of target protein and associated ligand.

FIG. 4 is an illustrative example of associated probability distributions of peptide ligand's amino acid residue primary voxels.

FIG. 5 is an illustrative example of a signaling pathway specific discriminative classifier (SPS-DC) neural network classification of a target protein as being in an agonist-bound conformation.

FIG. 6 is an illustrative example of a signaling pathway specific discriminative classifier (SPS-DC) neural network classification of a target protein as being in an antagonist-bound conformation.

FIG. 7 is a schematic illustration of an example of an amino acid embedding neural network training procedure.

FIG. 8 is an illustrative example of a Class Activation Map (CAM) in an SPS-DC neural network classifying a target protein as being in an unbound conformation.

FIG. 9 is a schematic illustration of an example of a CAM mechanism showing discriminative feature map summation.

FIG. 10 is an illustrative example of a transformer architecture-based signaling pathway specific discriminative classifier (SPS-DC) neural network.

FIG. 11 is a schematic illustration of an example of a CAM-guided structure update engine.

FIG. 12 is an illustrative example of a training architecture of a bicapitate CAM-guided expert transformer for peptide ligand design given target protein sequence and structure.

FIG. 13 is an illustrative example of an inference architecture of a bicapitate CAM-guided expert transformer for peptide ligand sequence, structure, and docking site determination given target protein sequence and structure.

FIG. 14 is an illustrative example of a training architecture of a bicapitate CAM-guided expert transformer for small molecule drug ligand identity and docking site determination given target protein sequence and structure.

FIG. 15 is an illustrative example of an inference architecture of a bicapitate CAM-guided expert transformer for small molecule drug ligand identity and docking site determination given target protein sequence and structure.

FIG. 16 is a flow schematic of steps for one embodiment of a CAM-Guided structure refinement engine.

FIG. 17 is a flow schematic of steps for one embodiment of a bicapitate CAM-guided expert transformer inference engine.

FIG. 18 is an example of a computing environment.

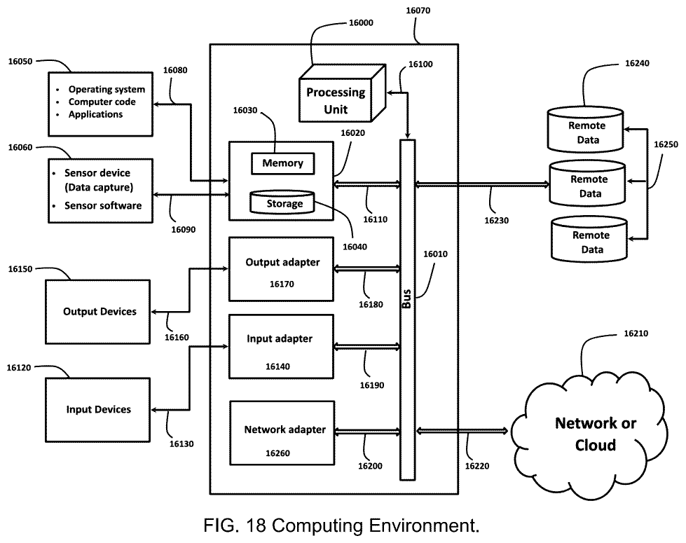

### DETAILED DESCRIPTION OF THE PREFERRED EMBODIMENTS

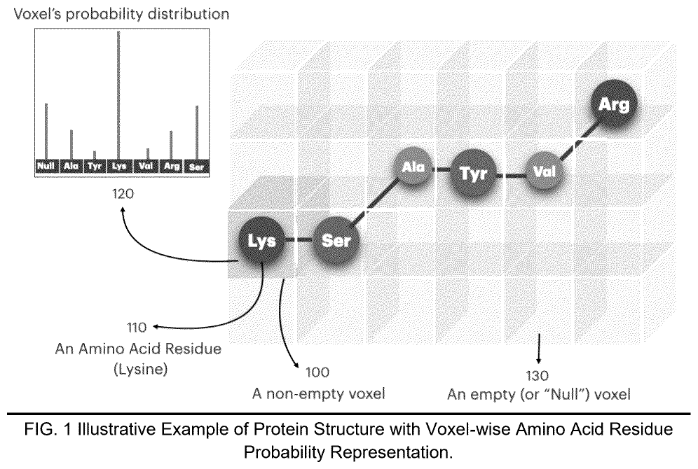

The illustration in FIG. 1 is a preferred embodiment of a volumetric probability representation of a protein structure. In this example, the first amino acid residue 110 is lysine and it is contained primarily in the non-empty voxel 100. The voxel's associated probability distribution 120 is illustrated and the domain consists of the protein's constituent amino acids {Lys, Ser, Ala, Tyr, Val, Arg} and {Null} for empty. As expected, the probability that the voxel holds lysine is higher than the probability that it is empty or that it holds any other of the constituent amino acids.

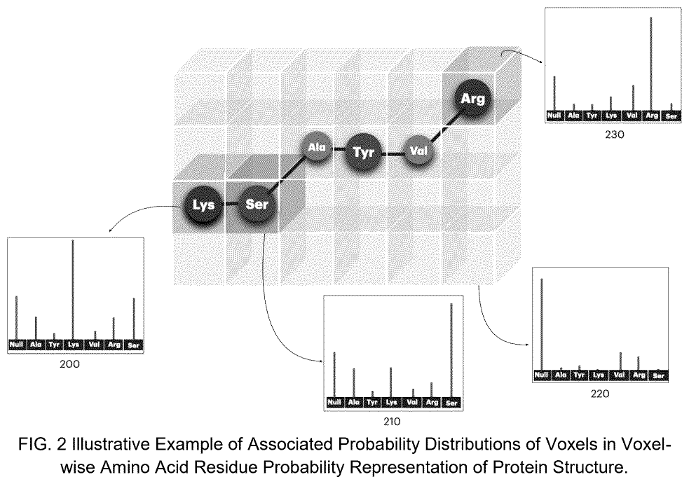

FIG. 2 illustrates the same protein as FIG. 1, here, in addition to the probability distribution of the primary voxel of the lysine residue 200, also depicted are the probability distributions for the primary voxel of the serine residue 210, the primary voxel of the arginine residue 230, and a primarily empty voxel 220. The neighborhood information is reflected for instance in the primarily serine voxel 210 which has a significant probability of being empty or of instead containing the lysine residue.

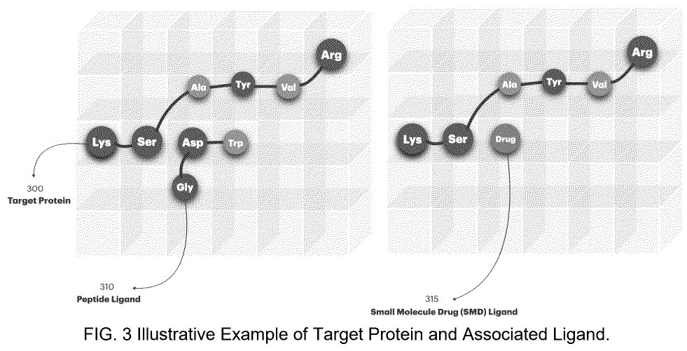

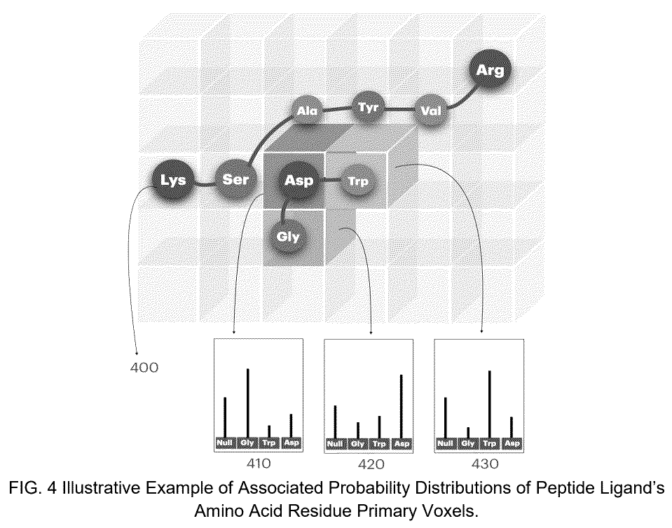

FIG. 3 illustrates a target protein 300, an associated oligopeptide ligand 310 consisting of three amino acids, and a small molecule drug 315. Further, FIG. 4 depicts the probability distributions 410, 420, and 430 of each of the three primary voxels of the oligopeptide ligand's three amino acids: aspartic acid, glycine, and tryptophan respectively.

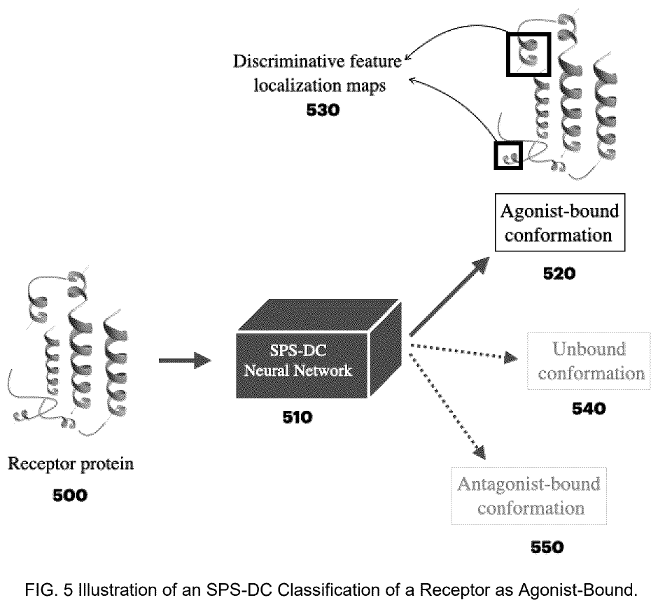

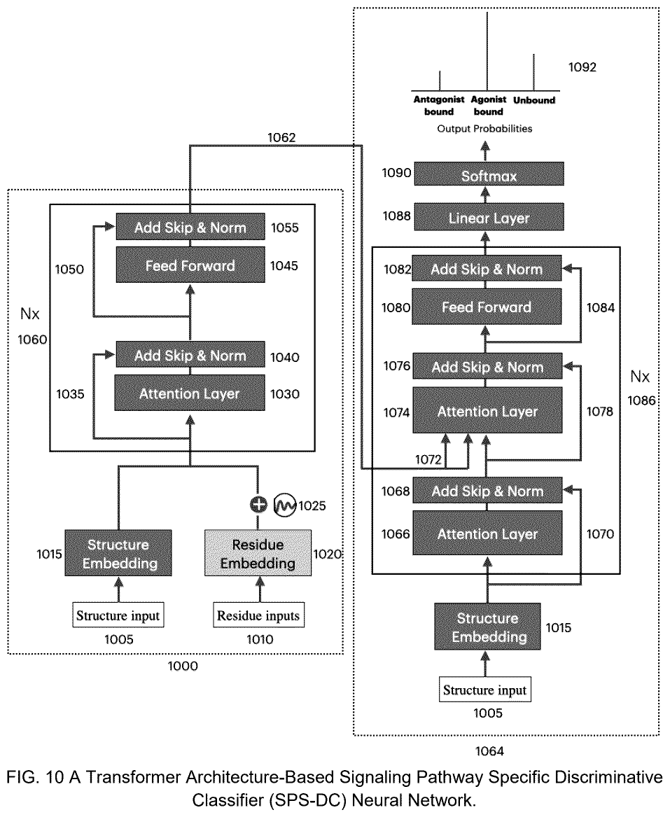

The exemplary illustration in FIG. 5 depicts a receptor protein structure representation 500 passed as input into an SPS-DC neural network 510 which classifies the input receptor protein structure as being of the agonist-bound conformation 520. Furthermore, the SPS-DC localizes the discriminative feature maps 530. The unbound conformation 540 and antagonist-bound conformation 550 are also shown. The SPS-DC neural network 510 may be a graphical neural network, a graphical convolutional neural network, a convolutional neural network, a recurrent neural network, a transformer-based network architecture as illustrated in FIG. 10 below, or it may be any other neural network configuration or architecture that enables representation of target proteins in a space where meaningful ligand effect classification can be conducted.

Training of the SPS-DC neural network 510 relies on a database of target protein-ligand complexes. The database includes multi-dimensional indexing across associated attributes including but not limited to signaling pathway specifiers and ligand effect category. In particular, each of the possible values of each categorization random variable should be represented in a statistically representative manner in the database. For instance, as depicted in FIG. 5, consider a simple example of a receptor with primarily three stable structural conformations at equilibrium (e.g. a ‘agonist-bound,’ ‘antagonist-bound,’ and ‘unbound’). The target protein-ligand complexes database for training the SPS-DC should contain a diverse plurality of representations of target protein-ligand complexes in their respective agonist-bound conformations, a diverse plurality of representations of target protein-ligand complexes in their respective antagonist-bound conformations, and a diverse plurality of representations of target proteins in their respective unbound conformations. Furthermore, the database should be sufficiently large and sufficiently diverse to encode a learnable representative pattern which the SPS-DC 510 can effectively learn.

After training, given as input a target protein structure representation previously unseen to the SPS-DC neural network, it outputs its classification prediction (i.e. agonist-bound conformation vs antagonist-bound conformation vs unbound conformation). In the example depicted in FIG. 5, the SPS-DC is a trinary classifier. However, the SPS-DC may be n-ary where n is simply the number of ligand effects classes of the particular application.

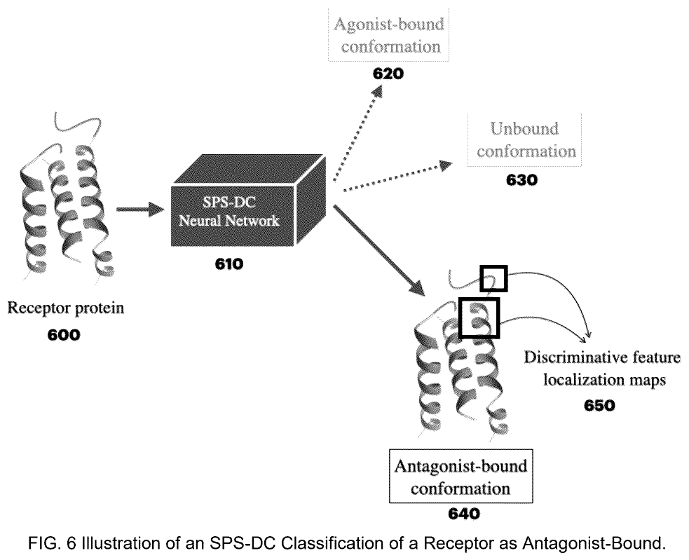

The exemplary illustration in FIG. 6 depicts a receptor protein structure representation 600 passed as input into an SPS-DC neural network 610 which classifies the input receptor protein structure as being in an antagonist-bound conformation 640. The discriminative feature maps 650 accompany the classification output.

FIG. 5 and FIG. 6 both depict an example whereby the SPS-DC neural network is a trinary classifier that has been trained such that given a target protein structure representation, it infers whether that input structure is of the agonist-bound vs antagonist-bound vs unbound class. Of note, the SPS-DC neural network may be trained instead to infer other properties from receptor protein structure. The property (or properties) which the SPS-DC is trained to evaluate depend on the objective. The training database simply needs to be representative of the property values, and must contain a sufficiently large and sufficiently representative diverse plurality of structural conformations across property values.

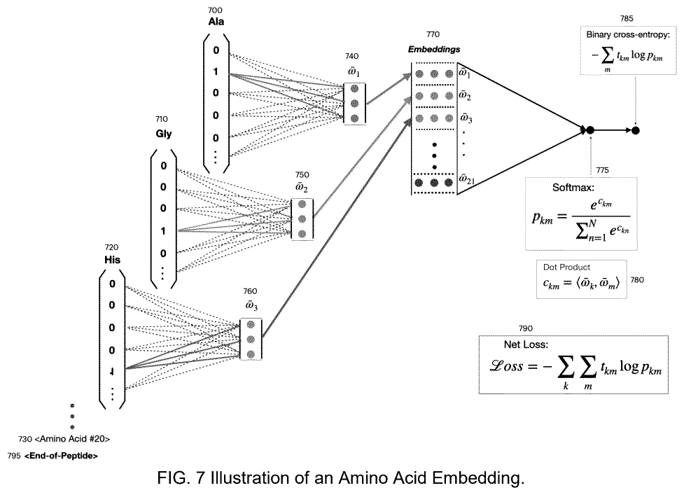

FIG. 7 illustrates the amino acid embedding procedure. The initial encoding of the amino acid residues is a one-hot-encoding as illustrated in 700, 710, and 720, wherein all but one entry of the vector are zeros and the non-zero entry is a 1 indicating the amino acid it encodes. The one-hot-encoding is sparse and does not convey any semantic meaning, serving instead only as a unique identifier of the respective amino acid.

In one embodiment of the invention, there are 20+n such one-hot-encoder vectors, where 20 are for the 20 amino acids in humans, and n is the number of auxiliary tokens such as <end-of-peptide>token 795.

Each of the one-hot-encoder vectors are used to right multiply a shared weight matrix, thereby effectively picking out the one column of the shared weight matrix that corresponds to the unique index or address of that amino acid. That unique column is the corresponding vector embedding of that amino acid, as illustrated in 740, 750, and 760, corresponding respectively to one-hot-encoder vectors 700, 710, and 720 respectively. As noted, since the vector embeddings are simply columns of the shared weight matrix, it follows that they are themselves the learnable weights of the residue embedding neural network.

The residue embedding neural network takes the pairwise dot product 780 of embeddings. Then for each amino acid residue, it applies a softmax activation 775 to convert the vector of dot products into a probability distribution. In one embodiment, the probability distribution is intended to indicate the probability that the subject amino acid is in close sequence proximity to the amino acid being evaluated. If they are typically in close proximity, then the dot product of their respective embedding vectors should be closer to 1, and if they are rarely in close sequence proximity, the dot product should be closer to zero. There are other methods for implementing the loss function 790 in this invention, sequence proximity being just a non-limiting example.

In one embodiment, a cross entropy loss 785 can then be used, wherein the target distribution is empirically determined by sequence proximity, i.e. tₖₘ is a distribution whose value is closer to 1 for amino acids m typically of close sequence proximity to amino acid k, and closer to zero for amino acids m of far sequence proximity to amino acid k. The net loss 790 is the sum of the losses across all the amino acids. By way of example but not limitation, an optimization method such as stochastic gradient descent can then be used to train the network.

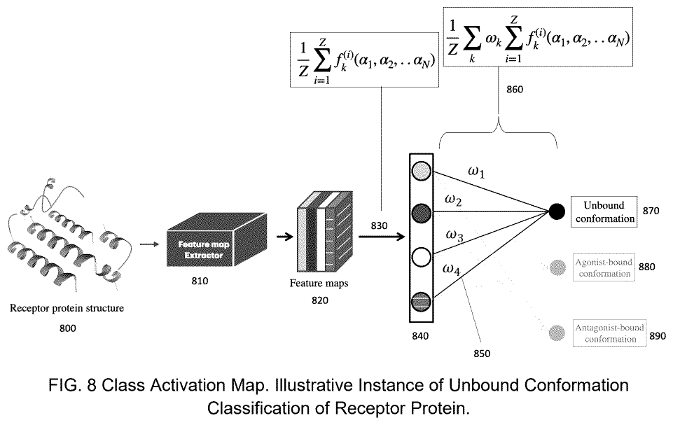

FIG. 8 depicts a preferred embodiment of a discriminative feature localization method of an SPS-DC neural network. In this example, the discriminative feature localization method is a Class Activation Mapping (CAM) and the SPS-DC has a Global Average Pooling (GAP) layer 830 for CAM. The feature extraction layers 810 act on an input target protein structure representation 800 to yield a set of feature maps 820. For each feature map, its values fₖ (α₁, . . . , α_{N}) are globally averaged as shown in 830 to yield a single entry in the feature vector 840. Where fx (α₁, . . . , α_{N}) is the kth feature map, αₚ is the pth axis variable of the feature map's domain wherein the pth axis is of dimension dim(αₚ). The total number of elements in each feature map is denoted Z and is given by,

$$Z = {\prod }_{p = 1}^{N}$$

$$dim ( {α}_{p} )$$

The feature vector 840 has as many entries as there are feature maps in the preceding layer 820. The feature vector is connected via a dense layer to the output scores as shown in 860. An activation function such as ReLU may be applied to the output scores, the higher of which is the output classification as illustrated in 870. It follows that since ReLU is monotonically increasing over the positive domain, the higher of the three outputs necessarily has the higher raw score computed via the formula in 860. One may factor in the weights ωₖ as follows:

$${\sum }_{k} ω k ( \frac{1}{Z} {\sum }_{i = 1}^{Z} {f}_{k}^{( i )} ( {α}_{1} , … , {α}_{N} ) ) = \frac{1}{Z} {\sum }_{k} {\sum }_{i = 1}^{Z} ( {ω}_{k} {f}_{k}^{( i )} ( {α}_{1} , … , {α}_{N} ) )$$

whereby ωₖfₖ(α₁, . . . , α_{N}) are weighted feature maps for which given a ReLU-like activation only the non-negative values contribute towards the classification and they do so proportionally, i.e. the higher the value of a point in the weighted feature map, the more it contributes towards the final output score of its associated class. This property is preserved through the global average pooling of the weighted feature maps. Summing over the k weights associated with a given class is equivalent to a discriminative map overlay.

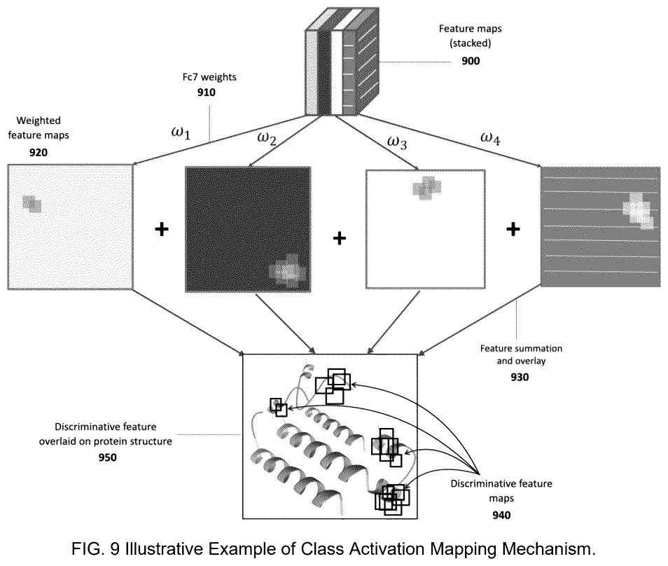

FIG. 9 elucidates a class activation mapping mechanism embodiment of discriminative feature localization. The final layer weights—i.e. the weights by which the feature maps are scaled—are shown in 910, yielding the weighted feature maps 920. The weighted feature maps are then upsampled back to the size of the original input target protein structure representation data 930 and are all overlaid on the representation as shown in 950. The discriminative feature maps 940 are directly superimposed on the input data after upsampling.

FIG. 10 is an illustration of a transformer architecture-based SPS-DC neural network embodiment. As noted earlier, the SPS-DC neural network can take any number of forms including but not limited to graphical neural network, graphical convolution neural network, convolutional neural network, transformer architecture, etc. In this embodiment of a transformer SPS-DC, an encoder-decoder architecture is shown. The encoder part 1000 accepts a structure input vector 1005 into the structure embedding 1015. The structure input vector is a vector of structure parameters. It is of fixed length, L, and zero padding is used for target proteins whose structure parameters are represented by a vector of smaller length and the fixed length, L. The fixed length, L, is a hyperparameter.

The structure embedding is a weight matrix, Ws, which the structure input vector, x, 1015 multiplies to yield the structure embedding vector, s, as follows:

WsX=S
where is an m×L matrix, where L is the fixed length of the structure input vector and m is the length of the amino acid residue embedding vectors. Both m and L are hyperparameters of the model.

The target protein's amino acid residue inputs 1010 can be in the form of one-hot-ecoder vectors which are passed into the residue embedding 1020 described in FIG. 7. A position encoding 1025 can be added to the output residue embedding vectors to imprint a signal of sequence position on the respective residue embeddings.

An array of vectors consisting of the structure embedding vector and each of the residue embedding vectors is passed as input into an attention layer 1030. The attention layer consists of three types of weight matrices, a query weight matrix, W_{q}, a key weight matrix, Wₖ, and a value weight matrix, Wᵥ. Each of the embedding vectors in the array are then multiplied by each of the three matrices to obtain respective queries, keys, and values, as follows:

W_{q}u=q

Wₖu=k

Wᵥu=V
where u is an embedding vector (i.e. either the structure embedding vector s or one of the residue embedding vectors r).

For each embedding vector in the array, its respective query vector is dot produced with the key vectors of all tokens in the array. Here we use the terms ‘token’ and ‘vector’ interchangeably to denote members of the array. Next a softmax operation is done on the resulting array to yield a probability distribution for each token. Next, for each token, a linear combination of values vis taken wherein the coefficient of each value is the respective probability. The output of this linear combination is then taken as the token's respective output into the next layer of the transformer. This is done for each token, therefore the length of the input array and the length of the output array from this attention layer 1030 are the same. Given the ith token, its corresponding coefficient associated with the jth token can be denoted cᵢⱼ and is given by,

$${c}_{i j} = \frac{{e}^{< qi , kj >}}{{\sum }_{p} {e}^{< qi , kp >}}$$

The attention layer output of the ith token can be denoted oᵢ and is then given by,

oᵢ=Σⱼcᵢⱼvⱼ

In some embodiments the dot product <qᵢ, kⱼ>can be scaled by a variance factor.

The array of outputs o; are then passed into a normalization layer 1040. Furthermore, a copy of the input array which was passed into the attention layer is passed into 1035 and added to the normalization layer, skipping the attention layer. This skip connection serves to preserve the pre-attention layer character signal thereby enhancing available signals for learning.

The output from the Add skip & norm layer 1040 is passed into a feed forward neural network layer 1045 and from there into another Add skip & norm layer 1055. The block module 1060 of “attention→add skip & norm-feed forward→Add skip & norm” is repeated N number of times where N is a hyperparameter of the model architecture.

The final output array of the encoder part is then passed 1062 into the decoder part 1064 In particular, it enters the decoder at a cross attention layer 1072, wherein the encoder output array joins the incoming token from the preceding layer 1068 of the decoder. The subject token then attends to all elements in the combined array via the previously described attention mechanism, hence the term cross attention.

The decoder receives input both from the encoder via cross attention input 1072 as well as directly via the structure vector input 1005. It enters a self-attention layer 1066 whose context array consists of only one token, initially the structure embedding vector, which self-attends to itself; after which it is passed to add skip & norm layer 1068 and then onwards to cross attention layer 1074. The block module 1086 repeats N times where N is a hyperparameter of the model.

The final output from the N repeated blocks 1086 is passed into a linear layer 1088 of length equal to the number of ligand effect classes. The linear layer can be connected to a discriminative feature localization mechanism. The output of the linear layer is then acted on by a softmax activation 1090 to generate a probability distribution 1092.

During training, in one embodiment, the probability distribution 1092 can be compared against a target distribution of labels using a cross entropy loss function. Then an optimization method such as stochastic gradient descent, for example, can be used to train the model. During inference, given a target protein sequence and structure representation, the model outputs a probability distribution 1092 predicting the ligand effect classification.

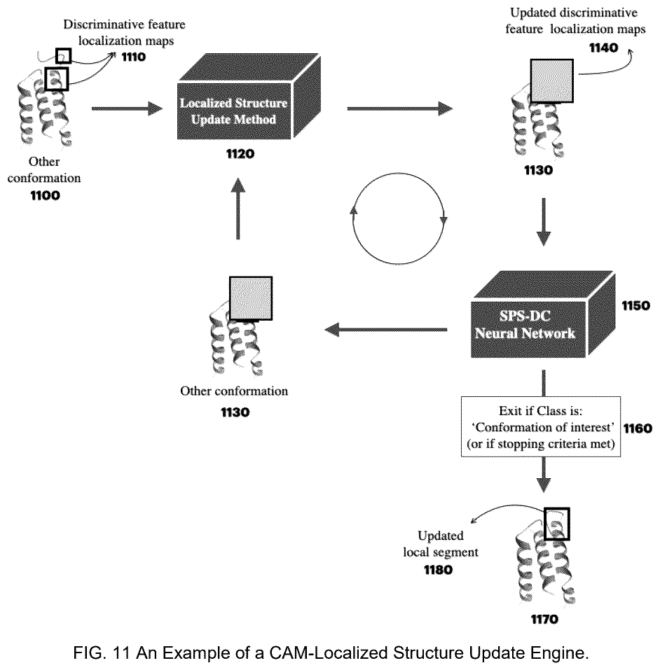

An exemplary embodiment of a Localized Structure Update Engine is depicted in FIG. 11. It involves as components, a localized structure update method 1120 and a trained SPS-DC neural network 1150. A target protein's structure representation SPS-DC-labeled as belonging to some ‘other conformation’ is passed in along with its discriminative feature maps 1110 as input into the localized structure method 1120. In one embodiment, the localized structure method acts on the input to locally update only the aspects indicated by the discriminative feature maps. This results in an updated local segment 1140. The updated target protein structure 1130 is then passed as input into the SPS-DC 1150. If the updated structure 1130 is found to be of the ‘conformation of interest’ as desired, the engine exits and outputs the updated structure representation 1170 along with a mask of the updated aspects 1180. If however, the resulting class is still the ‘other conformation’ and the stopping criteria (e.g. max number of iterations) is not yet met, then the updated structural conformation representation 1130 is passed as input into the localized structure update method 1120. The process continues in a loop as described till exit condition 1160 is met. When exit condition 1160 is met, the engine exits and outputs the representation of the most updated structural conformation 1170.

The localized structure update method 1120 could be implemented in any number of ways including but not limited to stochastic gradient descent and its variants, genetic algorithms and their variants, particle swarm optimization methods and their variants, simulated annealing methods and their variants, and Monte Carlo Tree Search (MCTS) methods and their variants. The key principle is simply to progressively move the SPS-DC score towards the conformation of interest and away from the other conformation. The progression of the score change need not be monotonic, either, but simply needs to progress in an expectation sense. For instance, random walk with pull type schema may not progress monotonically, but in aggregate (i.e. in an expectation sense) they progress in the correct direction.

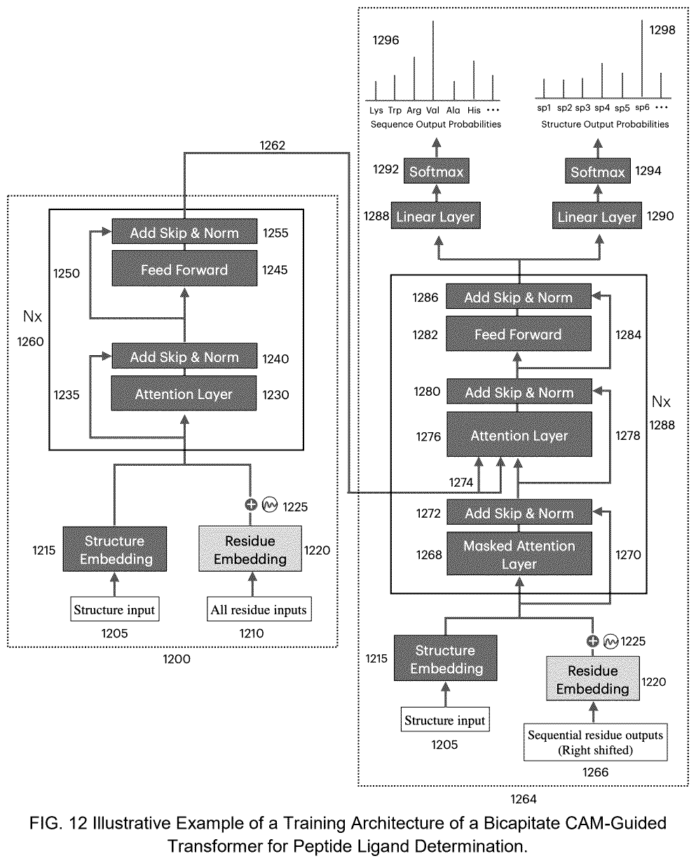

FIG. 12 is an illustrative example of a training architecture of a bicapitate CAM-guided expert transformer for peptide ligand sequence, structure, and docking site determination given a target protein sequence and structure representation. The encoder block 1200 is identical in form to the encoder block 1100 of the transformer-based SPS-DC neural network example described above in FIG. 11. The decoder block 1264, however, has a number of distinct aspects. Its final output is bicapitate in that it has two distinct heads, a sequence head and a structure head, terminating in output probability distributions 1296 and 1298 respectively. Its direct input consists of both a target protein structure input vector 1205 as well as a ligand residue input vector 1266 which enters sequentially in an autoregressive manner.

In some other embodiments as earlier mentioned, the emerging ligand's structure may also be autoregressively entered as a direct input.

The transformer training architecture is designed for parallelism. In particular, for each amino acid residue token in a peptide ligand sequence to be generated, the preceding amino acid residues of the ligand as well as the label (i.e. the correct amino acid residue token) are both known and available for end-to-end differentiable supervised learning. Hence the prediction of each amino acid residue token can be run simultaneously with the shared weights of the architecture being updated simultaneously. The implementation of this is reflected in the masked attention layer 1268, wherein for any given residue in the ligand sequence, the preceding tokens of the ligand are visible to the prediction algorithm and used in attention layer, but its residue answer label (i.e. the correct next amino acid in the sequence) is masked from the prediction algorithm.

End-to-end stochastic gradient descent, for example (or other optimization), then proceeds in parallel for each amino acid, wherein each parallel process updates the set of shared weights as it proceeds. This however, is simply an implementation embodiment example, and not a limitation in any way.

In the embodiment of FIG. 12, the <start-of-sequence>token is taken as the structure input vector 1205. Subsequent tokens are the amino acid residues and are passed in from the final output layer in an autoregressive manner. As noted however, since both the preceding residues of the ligand and the residue answer labels are fully known during training, the architecture is such that training can be done in parallel i.e. without needing to wait in sequence.

The residue embedding 1220 is as described earlier in FIG. 7.

The sequence head's final layer output probabilities 1296 are over the amino acids and auxiliary tokens such as an<end-of-sequence>token. By way of example but not limitation, a cross-entropy loss function can be implemented and then stochastic gradient descent (or other optimization) used to optimize the model. Therefore, backpropagation of errors computed at the sequence head terminal results in weight updates in the sequence head as well in all other upstream weights in the transformer body that contributed to the sequence head loss. In this sense, the non-capitate weights are shared.

Similarly, the structure head's final layer output probabilities 1298 are over the structure parameters for encoding a residue. By way of example but not limitation, they may be spatial coordinate locations of the voxels in a 3D grid, or they may be unique identifiers (“address”) of the voxels in a 3D grid, or representative values of a discretization of the range of possible torsion angles. Similarly to the sequence head, by way of example but not limitation, a cross-entropy loss function can be implemented and then stochastic gradient descent (or other optimization) used to optimize the model. Therefore, backpropagation of errors computed at the structure head terminal results in weight updates in the structure head as well in all other upstream weights in the transformer body that contributed to the structure head loss. In this sense, the non-capitate weights are shared.

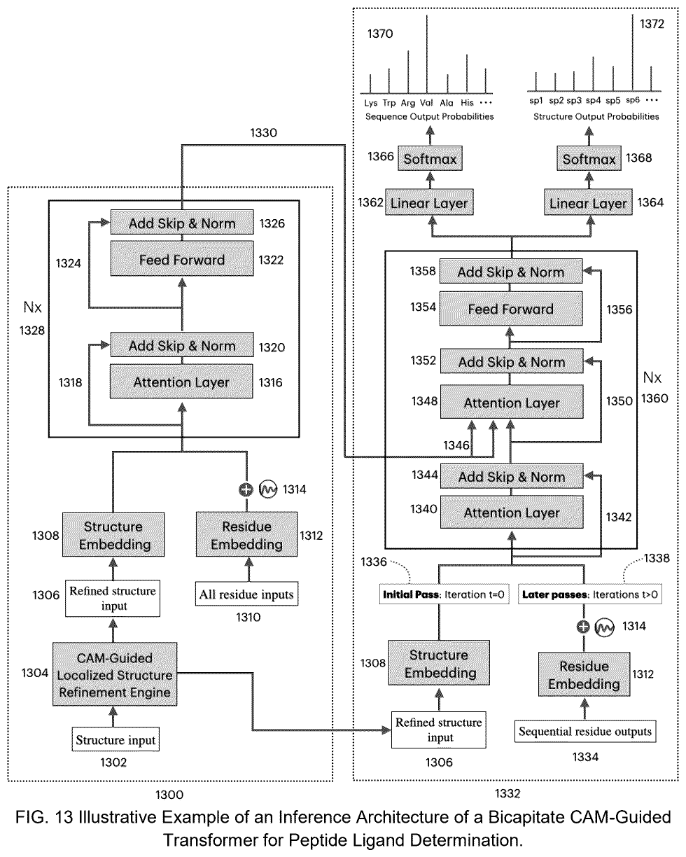

FIG. 13 is an illustrative example of an inference architecture of a bicapitate CAM-guided expert transformer for peptide ligand sequence, structure, and docking site determination given target protein sequence and structure. While FIG. 12 illustrates the training architecture, FIG. 13 illustrates the corresponding inference architecture. Here, the primary difference between training and inference is that during training both the preceding residues of the ligand and the subject residue answer label are known, while during inference only the preceding ligand residues are known, the residue answer label is not known. In both instances, training and inference, the target protein sequence and structure are known.

Therefore, for the inference architecture, the initial attention layer 1340 of the decoder 1332 is not a masked attention layer as it was in the case of the training architecture. Furthermore, an autoregressive process is needed because the output token of iteration t is the residue input subject token for iteration t+1. Furthermore, the <start-of-sequence> (i.e. t=0) subject token is the structure input vector.

In one embodiment, another critical distinction between training and inference is that in the inference architecture, the structure input 1302 is first passed into a CAM-guided localized structure refinement engine 1304, for refinement towards the desired ligand effect classification, i.e. towards the expertise of the transformer. The CAM-guided localized structure refinement engine 1304, was earlier described in FIG. 11. Given a structure input vector 1302, it is passed through the respective layers of the CAM-guided transformer expert, ultimately yielding at its sequence head an amino acid residue, the first in the ligand sequence, and yielding at its structure head the structure parameters of that first amino acid residue. This residue is then passed in as input 1334, proceeds through the indicated layers, and yields the second amino acid residue, and so on till an<end-of-peptide>token is reached, at which point the process halts and the generated ligand peptide sequence, structure, and docking site are returned as the final output.

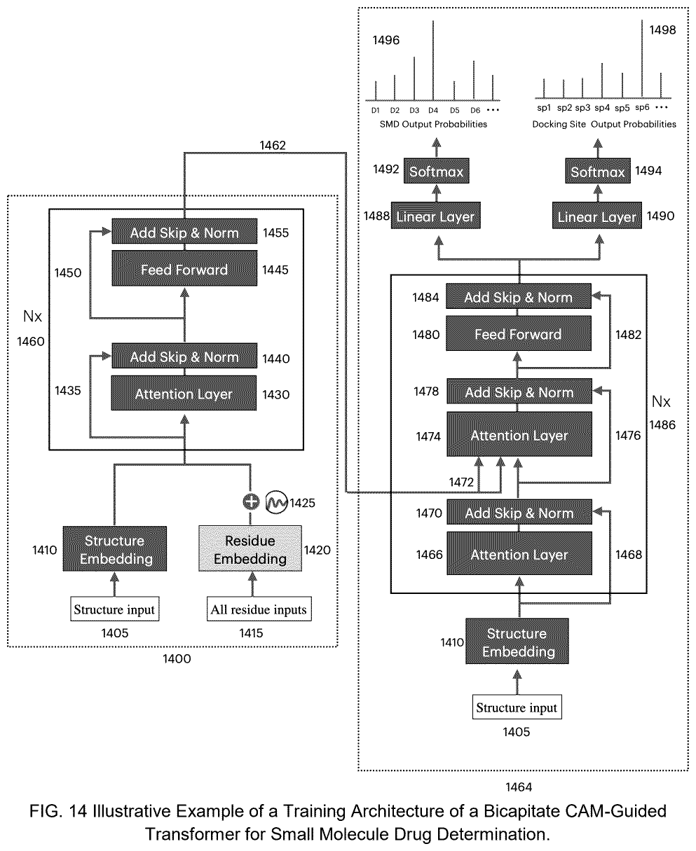

FIG. 14 is an illustrative example of a training architecture of a bicapitate CAM-guided expert transformer for small molecule drug ligand identity and docking site determination given target protein sequence and structure. In this case the ligand sequence is of length 1, so there is only need for a<start-of-sequence>token which here again is the structure input vector 1405. Unlike in the case of peptide ligand design training FIG. 12, there is no ligand residue input since the sequence here is of length 1. For the same reason, the initial attention layer 1466 is not masked. The sequence head's final output probability distribution 1496 is over a library of Small Molecule Drug (SMD) ligand candidates, while the structure head's final output probability distribution 1498 is over the possible structure parameter values of the SMD. However, since the SMD is a ligand of sequence length 1, we take structure specification to be the location of the SMD relative to the target protein, for instance the “address” of the voxel where the SMD resides within a 3D grid. Other aspects of the training architecture are the same as for the peptide ligand design instance, FIG. 12.

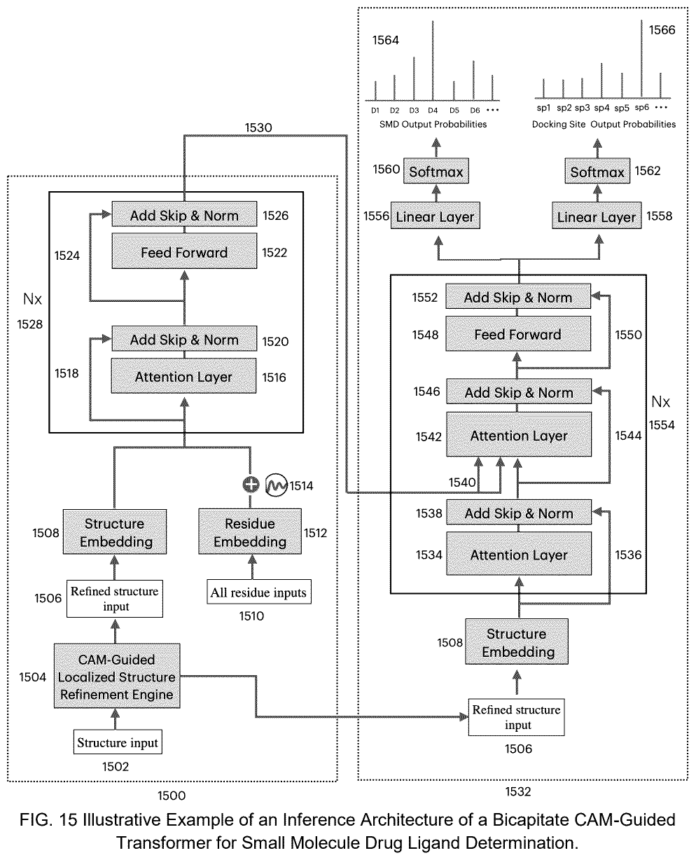

FIG. 15 is an illustrative example of an inference architecture of a bicapitate CAM-guided expert transformer for small molecule drug ligand identity and docking site determination given target protein sequence and structure. The inference architecture, FIG. 15, differs from its corresponding training architecture, FIG. 14, in that the structure input vector 1502 is first passed into a CAM-guided localized structure refinement engine 1504, for refinement towards the desired ligand effect classification, i.e. towards the expertise of the transformer. The CAM-guided localized structure refinement engine 1504, was earlier described in FIG. 11. The resulting refined structure input 1506 is what is then passed into the structure embedding module 1508. As input, the architecture accepts the structure 1502 and sequence 1510 of a target protein; and returns from its “sequence head” a probability distribution 1564 over candidate small molecule drugs (SMDs), and from its “structure head” a probability distribution 1566 over structure parameters encoding the possible locations (i.e. docking sites) of the SMD.

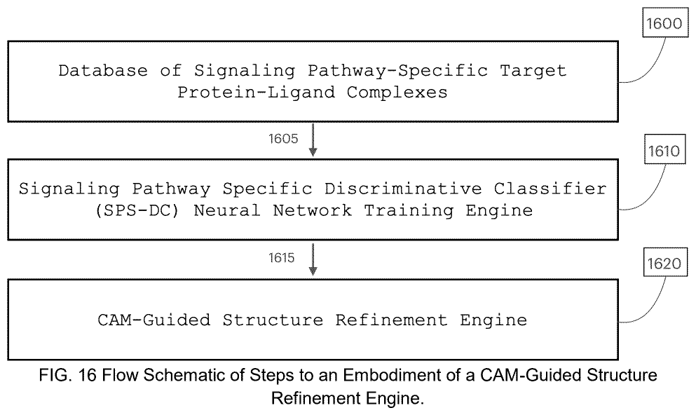

FIG. 16 is a flow schematic of steps to an embodiment of a CAM-guided structure refinement engine. The SPS-DC neural network training engine 1610 used a signaling pathway specific database of target protein-ligand complexes 1600. In turn, the CAM-guided structure refinement engine 1620 has the trained SPS-DC neural network as a main component.

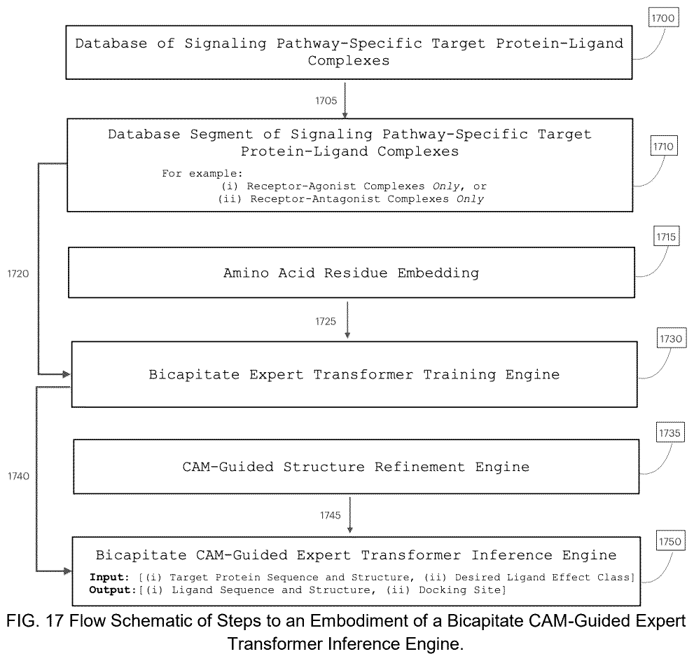

FIG. 17 is a flow schematic of steps to an embodiment of a bicapitate CAM-guided expert transformer inference engine. The database of signaling pathway specific target protein-ligand complexes 1700 is further segmented by ligand effect class to get a segmented database 1710. This in turn is used in the expert transformer training engine 1730. The trained amino acid residue embedding 1715 is also an important component of the expert transformer training engine 1730. The CAM-guided structure refinement engine described in FIG. 11, is a component of the bicapitate CAM-guided expert transformer inference engine 1750. The bicapitate CAM-guided expert transformer inference engine takes as input a target protein structure and sequence, and also takes the desired ligand effect class (e.g. agonist, antagonist, etc), and yields as output the ligand sequence, structure, and docking site.

Ones with ordinary skill in the art will recognize that the invention disclosed herein can be implemented over an arbitrary range of computing configurations. We will refer to any instantiation of these computing configurations as the computing environment. An illustrative example of a computing environment is depicted in The Computing Environment FIG. Examples of computing environments include but are not limited to desktop computers, laptop computers, tablet personal computers, mainframes, mobile smart phones, smart television, programmable hand-held devices and consumer products, distributed computing infrastructures over a network, cloud computing environments, or any assembly of computing components such as memory and processing—for example.

As illustrated in The Computing Environment FIG, the invention disclosed herein can be implemented over a system that contains a device or unit for processing the instructions of the invention. This processing unit 16000 can be a single core central processing unit (CPU), multiple core CPU, graphics processing unit (GPU), multiplexed or multiply-connected GPU system, or any other homogeneous or heterogeneous distributed network of processors.

In some embodiment of the invention disclosed herein, the computing environment can contain a memory mechanism to store computer-readable media. By way of example and not limitation, this can include removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer.

As depicted in The Computing Environment FIG, the computing environment can include a system memory 16030 which can be volatile memory such as random access memory (RAM) and may also include non-volatile memory such as read-only memory (ROM). Additionally, there typically is some mass storage device 16040 associated with the computing environment, which can take the form of hard disc drive (HDD), solid state drive, or CD, CD-ROM, blu-ray disc or other optical media storage device. In some other embodiments of the invention the system can be connected to remote data 16240.

The computer readable content stored on the various memory devices can include an operating system, computer codes, and other applications 16050. By way of example not limitation, the operating system can be any number of proprietary software such as Microsoft windows, Android, Macintosh operating system, iphone operating system (iOS), or Linux commercial distributions. It can also be open source software such as Linux versions e.g. Ubuntu. In other embodiments of the invention, data processing software and connection instructions to a sensor device 16060 can also be stored on the memory mechanism. The procedural algorithm set forth in the disclosure herein can be stored on—but not limited to—any of the aforementioned memory mechanisms. In particular, computer readable instructions for training and subsequent image classification tasks can be stored on the memory mechanism.

The computing environment typically includes a system bus 16010 through which the various computing components are connected and communicate with each other. The system bus 16010 can consist of a memory bus, an address bus, and a control bus. Furthermore, it can be implemented via a number of architectures including but not limited to Industry Standard Architecture (ISA) bus, Extended ISA (EISA) bus, Universal Serial Bus (USB), microchannel bus, peripheral component interconnect (PCI) bus, PCI-Express bus, Video Electronics Standard Association (VESA) local bus, Small Computer System Interface (SCSI) bus, and Accelerated Graphics Port (AGP) bus. The bus system can take the form of wired or wireless channels, and all components of the computer can be located remote from each other and connected via the bus system. By way of example and not of limitation, the processing unit 16000, memory 16020, input devices 16120, output devices 16150 can all be connected via the bus system. In the representation depicted in The Computing Environment FIG, by way of example not limitation, the processing unit 16000 can be connected to the main system bus 16010 via a bus route connection 16100, the memory 16020 can be connected via a bus route 16110, the output adapter 16170 can be connected via a bus route 16180, the input adapter 16140 can be connected via a bus route 16190, the network adapter 16260 can be connected via a bus route 16200, the remote data store 16240 can be connected via a bus route 16230, and the cloud infrastructure can be connected to the main system bus vis a bus route 16220.

In some embodiment of the invention disclosed herein, The Computing Environment FIG illustrates that instructions and commands can be input by the user using any number of input devices 16120. The input device 16120 can be connected to an input adapter 16140 via an interface 16130 and/or via coupling to a tributary of the bus system 16010. Examples of input devices 16120 include but are by no means limited to keyboards, mouse devices, stylus pens, touchscreen mechanisms and other tactile systems, microphones, joysticks, infrared (IR) remote control systems, optical perception systems, body suits and other motion detectors. In addition to the bus system 16010, examples of interfaces through which the input device 16120 can be connected include but are by no means limited to USB ports, IR interface, IEEE 802.15.1 short wavelength UHF radio wave system (bluetooth), parallel ports, game ports, and IEEE 1394 serial ports such as FireWire, i.LINK, and Lynx.

In some embodiment of the invention disclosed herein, The Computing Environment FIG illustrates that output data, instructions, and other media can be output via any number of output devices 16150. The output device 16150 can be connected to an output adapter 16170 via an interface 16160 and/or via coupling to a tributary of the bus system 16010. Examples of output devices 16150 include but are by no means limited to computer monitors, printers, speakers, vibration systems, and direct write of computer-readable instructions to memory devices and mechanisms. Such memory devices and mechanisms can include by way of example and not limitation, removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer. In addition to the bus system 16010, examples of interfaces through which the output device 16150 can be connected include but are by no means limited to USB ports, IR interface, IEEE 802.15.1 short wavelength UHF radio wave system (bluetooth), parallel ports, game ports, and IEEE 1394 serial ports such as FireWire, i.LINK, and Lynx.

In some embodiment of the invention disclosed herein some of the computing components can be located remotely and connected to via a wired or wireless network. By way of example and not limitation, The Computing Environment FIG shows a cloud 16210 and a remote data source 16240 connected to the main system bus 16010 via bus routes 16220 and 16230 respectively. The cloud computing infrastructure 16210 can itself contain any number of computing components or a complete computing environment in the form of a virtual machine (VM). The remote data source 16240 can be connected via a network to any number of external sources such as NMR spectrometry devices, X-ray diffraction devices, electron microscopes, imaging devices, imaging systems, or imaging software.

In some embodiment of the invention disclosed herein, a sensor system 16060 which captures and pre-processes data is attached directly to the system. For example, this may be an electron microscope (and associated image processing software); it may be a camera in the case of an imaging system, say for processing distance map photographs; or it may be an X-ray crystallography machine or an NMR spectrometer (and associated software), excetera. Stored in the memory mechanism—16020, 16240, or 16210—are machine learning models, algorithms, and data products developed according to the procedures set-forth herein. Computer-readable instructions are also stored in the memory mechanism, so that upon command, protein structure representation data, its substrates and associated data can be captured or can be received over a network from a remote or local previously collated database. This transmission of data can be done over a wired or wireless network as previously detailed, as the source and/or recipient of the data output can be at a remote location.

The objects set forth in the preceding are presented in an illustrative manner for reason of efficiency. It is hereby noted that the above disclosed methods and systems can be implemented in manners such that modifications are made to the particular illustration presented above, while yet the spirit and scope of the invention is retained. The interpretation of the above disclosure is to contain such modifications, and is not to be limited to the particular illustrative examples and associated drawings set-forth herein.

Furthermore, by intention, the following claims encompass all of the general and specific attributes of the invention described herein; and encompass all possible expressions of the scope of the invention, which can be interpreted—as pertaining to language—as falling between the aforementioned general and specific ends.

What is claimed:

1. A method, comprising:
a. receiving, at a processor, a plurality of representations of target protein-ligand complexes;
b. using the plurality of representations of target protein-ligand complexes to train a neural network:
i. wherein the neural network is a transformer with multiple final output heads,
ii. wherein the transformer neural network is configured to accept the target protein's sequence and structure representation as input, and return an associated candidate ligand's sequence and structure as output,
iii, wherein one of the transformer's final output heads returns the candidate ligand's sequence as output and another of the transformer's final output heads returns the candidate ligand's structure as output;
c. using the trained transformer neural network to obtain the sequence and structure of a candidate ligand, given the sequence and structure of a target protein.

2. The method of claim 1, further comprising synthesizing the ligand.

3. The method of claim 1,
a. wherein the final layer of the transformer's structure head outputs a probability distribution over spatial locations;
b. wherein the final layer of the transformer's sequence head outputs a probability distribution over residues and an end-of-sequence token.

4. The method of claim 3, wherein the ligand sequence and structure generation are via an autoregressive process.

5. A method, as in the method of claim 4, for obtaining the sequence and structure of a candidate ligand, given a target protein's sequence and structure, wherein the method is also for obtaining an effective ligand's sequence and structure, the method further comprising:
a. randomly sampling the sequence head's output probability distribution to select the residue for each respective position in the ligand sequence during autoregression;
b. randomly sampling the structure head's output probability distribution to select the residue's spatial location for each respective position in the ligand sequence during autoregression;
c. stopping the autoregression iteration upon sampling the end-of-sequence token;
d. obtaining the resulting sequence of residue(s) and the respective corresponding spatial location(s) yielded by the autoregression process, and storing this output in memory as a candidate ligand's representation;
e. repeating the above process a plurality of times, each yielding the sequence and structure of a candidate ligand;
f. assessing the efficacy and interaction of each candidate ligand representation with the target protein representation;
g. selecting the most effective of the represented ligands from the plurality of represented candidate ligands.

6. The method of claim 5, further comprising synthesizing the ligand.

7. The method of claim 6, further comprising assessing the biological activity of the ligand in at least one of (α) in vitro and (b) in vivo.

8. The method of claim 7, wherein the target protein is a receptor, the ligand is a peptide ligand, and the ligand residues are amino acids.

9. The method of claim 7, wherein the target protein is a receptor, wherein the ligand is a small molecule drug, wherein the ligand residues are small molecule drugs, and wherein the ligand sequence is taken as being of length 1, i.e. each candidate ligand consists of a single small molecule drug.

10. An apparatus, comprising: a processor and an associated memory, wherein the memory stores instructions that when executed by the processor, cause the processor to:
a. receive representations of a plurality of target protein-ligand complexes;
b. use the plurality of representations of target protein-ligand complexes to train a neural network:
i. wherein the neural network is a transformer with multiple final output heads,
ii. wherein the neural network is configured to accept the target protein's sequence and structure representation as input, and return an associated candidate ligand's sequence and structure representation as output,
iii. wherein one of the transformer's final output heads returns the candidate ligand's sequence as output and another of the transformer's final output heads returns the candidate ligand's structure as output;
c. use the trained neural network to obtain the sequence and structure representation of a candidate ligand, given a target protein sequence and structure representation;
d. transmit instructions to synthesize the ligand.

11. A method, comprising:
a. receiving, at a processor, representations of a plurality of target protein-ligand complexes;
b. training a neural network to classify the plurality of target proteins:
i. wherein the neural network is equipped with a discriminative feature localization mechanism,
ii. wherein the classes encode the categories of ligand effect on the target protein,
iii. wherein the neural network is configured to accept the target protein's sequence and structure representation as input, and return the associated ligand's effect classification as output,
iv. wherein the neural network output also includes a discriminative feature localization map;
c. receiving, at a processor, a set of initial values of a plurality of structure parameters specifying the target protein's conformational structure;
d. using, via the processor, the trained neural network to perform inference on the initial values of the protein's conformational structure representation,
i, wherein the neural network outputs both the ligand effect classification and the discriminative feature map;
e. receiving, at a processor, a local structure update method, which is a set of instructions to update the values of the localized subset of structure parameters specified by the discriminative feature map,
i. wherein the local structure update method consists of a plurality of iterative steps, and some termination criteria,
ii. wherein the output of each iterative update step—an updated conformational structure representation—is evaluated by the neural network, yielding an updated classification score and an updated discriminative feature map,
iii. wherein:
1. if termination criteria are not yet met, then the updated conformational structure representation and the updated discriminative feature map are both re-entered as input into the local update method, else
2. if termination criteria are met, then the local structure update iteration terminates, and the updated conformational structure representation and the updated discriminative feature map are both returned as output;
f. selecting from the representations of a plurality of target protein-ligand complexes, a subset of complexes with a specific ligand effect category;
g. using the selected specific subset of complexes to train an expert neural network:
i. wherein the expertise of the neural network is the specific ligand effect category of its training dataset,
ii. wherein at inference, the local structure update method is first used to update the input structure representation of the target protein towards the expertise category of the expert neural network,
iii. wherein at inference, the expert neural network's action is on the updated structure representation of the target protein returned by the local structure update method,
iv. wherein the expert neural network is a multicapitate transformer, i.e. a transformer with multiple final output heads,
v. wherein the transformer is configured to accept the target protein's sequence and structure representation as input,
vi. wherein one of the transformer's final output heads returns the candidate ligand's sequence as output and another of the transformer's final output heads returns the candidate ligand's structure as output.

12. The method of claim 11, wherein the transformer architecture is of encoder-decoder type.

13. The method of claim 12, wherein the structure representation is acted on by a structure embedding whose weights are a subset of the learnable parameters of the transformer.

14. The method of claim 13, wherein the start-of-sequence vector input into the decoder is the target protein's structure embedding vector.

15. The method of claim 14, wherein the cross-attention context array includes the structure embedding vector of the target protein, and each of the residue embedding vectors, one per amino acid in the target protein sequence.

16. The method of claim 15, wherein the final layer of the transformer's sequence head outputs a probability distribution over the residues and an end-of-sequence token.

17. The method of claim 16, wherein the final layer of the transformer's structure head outputs a probability distribution over a set of possible spatial locations.

18. The method of claim 17, wherein the ligand sequence and structure generation are via an autoregressive process.

19. A method, as in the method of claim 18, for obtaining the sequence and structure of a candidate ligand of a specified effect category, given a target protein sequence and structure, wherein the method is also for obtaining an effective ligand's sequence and structure, the method further comprising:
a. randomly sampling the sequence head's output probability distribution to select the residue for each respective position in the ligand sequence during autoregression;
b. randomly sampling the structure head's output probability distribution to select the residue's spatial location for each respective position in the ligand sequence during autoregression;
c. stopping the autoregression iteration upon sampling the end-of-sequence token;
d. obtaining the resulting sequence of residue(s) and their corresponding spatial locations yielded by the autoregression process, and storing them in memory as a candidate ligand;
e. repeating the above process a plurality of times, each yielding the sequence and structure of a candidate ligand;
f. assesssing the efficacy and interaction of each candidate ligand with the target receptor;
g. selecting the most effective ligand from the plurality of candidate ligands.

20. The method of claim 19, further comprising synthesizing the ligand.
